(19) United States
(12) Patent Application Publication
(10) Pub. No.: US 2026/0345120 A1
(43) Pub. Date: Nov. 5, 2026

(54) ULTRASOUND INTRACAVITARY PROBE SUITABLE FOR PELVIC EXAMINATIONS, AND ULTRASOUND IMAGING DEVICE

(71) Applicant: SHENZHEN MINDRAY BIO-MEDICAL ELECTRONICS CO., LTD., Shenzhen (CN)

(72) Inventors: Rui SONG, Shenzhen (CN); Ming TANG, Shenzhen (CN)

(73) Assignee: SHENZHEN MINDRAY BIO-MEDICAL ELECTRONICS CO., LTD., Shenzhen (CN)

(21) Appl. No.: 19/714,246

(22) Filed: Jun. 19, 2026

Related U.S. Application Data
(63) Continuation of application No. PCT/CN2024/140077, filed on Dec. 17, 2024.

(30) Foreign Application Priority Data
Dec. 22, 2023 (CN) 202311786829.4

(51) Int. Cl. A61B 8/00; A61B 8/08; A61B 8/12

(52) U.S. Cl. CPC A61B 8/4461; A61B 8/08; A61B 8/12; A61B 8/4488; A61B 8/4494

(57) ABSTRACT

An ultrasound intracavitary probe and an ultrasound imaging device are provided. The ultrasound intracavitary probe includes a housing assembly, a driving assembly and a transducer assembly having a spindle. The transducer assembly includes at least two linear array transducers each including array elements. A probe housing includes an acoustic window and a connecting housing. The at least two linear array transducers are arranged in the acoustic window and spaced apart in the circumferential direction of the spindle. The at least two linear array transducers can transmit ultrasound waves to a first target area and receive returned ultrasound echoes to obtain at least two sets of ultrasound echo data corresponding to the at least two linear array transducers that is processed to obtain ultrasound images in at least two different longitudinal sections.

### CROSS-REFERENCE TO RELATED APPLICATION

This application is a Bypass Continuation Application of PCT/CN2024/140077 filed on Dec. 17, 2024, which claims priority to and the benefit of Chinese Patent Application No. 202311786829.4, filed on Dec. 22, 2023, the disclosure of which is incorporated herein by reference in its entirety.

### TECHNICAL FIELD

The present disclosure relates to medical examination device, and in particular to intracavitary ultrasound probes for pelvic examination and ultrasound imaging devices thereof.

### BACKGROUND

Intracavitary ultrasound probes are primarily used for intracavitary ultrasound examinations. During the examination, the transducer assembly on the spindle of the probe is driven by a built-in motor to scan a circumferential section within the cavity to obtain ultrasound images. When performing this circumferential scan, medical staff must hold the probe and remain stationary relative to the patient to obtain clearer ultrasound images. However, holding the probe for extended periods can cause wrist pain for medical staff and even discomfort and pain for the patient.

### SUMMARY

The present disclosure provides an intracavitary ultrasound probe and ultrasound imaging device for pelvic examination, which can obtain ultrasound images in at least two different longitudinal section through at least two linear array transducers mounted on the spindle, thereby shortening the time required for the circumferential scanning of the intracavitary ultrasound probe and avoiding shaking caused by prolonged holding during scanning, resulting in clearer ultrasound images.

In an embodiment, an intracavitary ultrasound probe for pelvic examination is provided, which may include a housing assembly, a driving assembly and a transducer assembly having a spindle, the housing assembly including a handle housing and a probe housing connected to the handle housing, the transducer assembly being arranged in the probe housing, and the driving assembly being arranged in the handle housing and connected to the spindle to drive the spindle to rotate about a central axis of the spindle;
where, the transducer assembly includes at least two linear array transducers, each of which includes multiple array elements linearly arranged along a preset direction of the spindle, the probe housing is provided with an acoustic window and a connecting housing, an end of the connecting housing facing away from the acoustic window being connected to the handle housing, and the at least two linear array transducers are spaced apart in a circumferential direction of the spindle and arranged in the acoustic window such that the spindle is able to drive the at least two linear array transducers to rotate around the central axis of the spindle;
and where, after being excited, the at least two linear array transducers transmit ultrasound waves in a radial direction of the spindle to a first target region in a pelvic cavity and receive returned ultrasound echoes to obtain at least two groups of ultrasound echo data corresponding to the at least two linear array transducers, and the at least two groups of ultrasound echo data are processed to obtain ultrasound images in at least two different longitudinal sections.

In an embodiment, the transducer assembly includes a first linear array transducer and a second linear array transducer, and where, an operating frequency range of the first linear array transducer is different from or the same as an operating frequency range of the second linear array transducer, and/or, a size of the first linear array transducer is different from or the same as a size of the second linear array transducer, and/or, a distance from a front end of the first linear array transducer to a front end of the spindle is different from or the same as a distance from a front end of the second linear array transducer to the front end of the spindle.

In an embodiment, the transducer assembly includes a first linear array transducer and a second linear array transducer, and the first linear array transducer and the second linear array transducer are symmetrically arranged on two side walls of the spindle with respect to a plane passing through the central axis of the spindle.

In an embodiment, the intracavitary ultrasound probe may further include a convex array transducer, where, the spindle includes a front end and a rear end extending along an axial direction of the spindle, the driving assembly is connected to the rear end of the spindle or to a position of the spindle near the rear end, and the convex array transducer is located at the front end of the spindle and is configured to transmit ultrasound waves to a second target region in the pelvic cavity and receive returned ultrasound echoes.

In an embodiment, an end of the acoustic window is provided with an opening, and the convex array transducer is arranged in the opening and sealed.

In an embodiment, the convex array transducer is mounted on the spindle and is able to be rotated about the central axis of the spindle; or, the intracavitary ultrasound probe further includes a rotating shaft that is able to be rotated independently relative to the spindle and the convex array transducer is connected to the rotating shaft.

In an embodiment, the transducer assembly includes a hinge shaft, the convex array transducer is hinged to the front end through the hinge shaft and is able to be oscillated around the hinge shaft, and an axis of the hinge shaft is perpendicular to the axis of the spindle.

In an embodiment, the convex array transducer is oscillated around the hinge shaft at an angle greater than 0° and less than or equal to 180°, the convex array transducer, after being excited, transmits ultrasound waves to the second target region in a radial direction of the hinge shaft and receives returned ultrasound echoes to obtain a convex array ultrasound echo data corresponding to the convex array transducer, and the convex array ultrasound echo data is processed to obtain ultrasound images of a tissue of the second target region in a transverse section.

In an embodiment, the housing assembly further includes a connecting seat with a spindle rotation hole, the connecting seat is connected between the handle housing and the connecting housing, one end of the spindle is rotatably mounted in the spindle rotation hole and is connected to the driving assembly, and the other end of the spindle extends into the acoustic window.

In an embodiment, the transducer assembly further includes a bearing, the bearing is arranged in the spindle rotation hole and the acoustic window, and the spindle passes through the bearing.

In an embodiment, intracavitary ultrasound probe may further include a limiting assembly, where the limiting assembly is mounted on at least one of the handle housing, the probe housing, the connecting seat and the spindle to enable the spindle to rotate at any angle within a range of not less than 720 degrees.

In an embodiment, the limiting assembly includes a fixing component, a limiting component and at least one connecting component, the fixing component is fixed to the housing assembly, the limiting component is fixed to the spindle, and the connecting component is rotatably mounted on the spindle and arranged between the fixing component and the limiting component.

In an embodiment, the driving assembly includes a driving component and a first reducer, and the driving component is connected to the spindle through the first reducer.

In an embodiment, the driving assembly further includes an encoder, and the encoder is arranged on a side of the driving component opposite to the first reducer and configured to detect a rotational speed of the spindle.

In an embodiment, a second reducer is connected between the encoder and the driving component, and a transmission ratio of the second reducer corresponds to a transmission ratio of the first reducer.

In an embodiment, a hollow cavity is formed in the spindle, a conductive layer for signal transmission is disposed in the hollow cavity, and the at least two linear array transducers are connected to a sidewall of the spindle and connected to the conductive layer;
and where, the linear array transducer includes a support frame, an acoustic lens, a backing layer and a piezoelectric layer electrically connected to the conductive layer, the acoustic lens is disposed outside the piezoelectric layer, the piezoelectric layer is disposed on the support frame through the backing layer, and the support frame is connected to the sidewall of the spindle.

In an embodiment, the preset direction is an axial direction of the spindle.

In an embodiment, an ultrasound imaging device is provided, which may include a display, an ultrasound host and an intracavitary ultrasound probe. The intracavitary ultrasound probe is connected to the ultrasound host.

The intracavitary ultrasound probe includes a housing assembly, a driving assembly and a transducer assembly having a spindle. The housing assembly includes a handle housing and a probe housing connected to the handle housing. The transducer assembly is arranged in the probe housing, and the driving assembly is arranged in the handle housing and connected to the spindle to drive the spindle to rotate about a central axis of the spindle.

The transducer assembly includes at least two linear array transducers, each of which includes multiple array elements linearly arranged along a preset direction of the spindle. The probe housing is provided with an acoustic window and a connecting housing. An end of the connecting housing facing away from the acoustic window is connected to the handle housing, and the at least two linear array transducers are spaced apart in a circumferential direction of the spindle and arranged in the acoustic window such that the spindle is able to drive the at least two linear array transducers to rotate around the central axis of the spindle.

After being excited, the at least two linear array transducers transmit ultrasound waves in a radial direction of the spindle to a first target region in the pelvic cavity and receive returned ultrasound echoes to obtain at least two groups of ultrasound echo data corresponding to the at least two linear array transducers.

The ultrasound host is configured to process the at least two groups of ultrasound echo data to obtain ultrasound images in at least two different longitudinal sections; and
the display is configured to display the ultrasound images.

The present disclosure provides intracavitary ultrasound probes and ultrasound imaging devices for pelvic examination. The intracavitary ultrasound probes comprise a housing assembly, a driving assembly and a transducer assembly with a spindle. The driving assembly is disposed inside the handle housing and is connected to the spindle for transmission. The driving assembly is configured to drive the spindle to rotate around its central axis, thereby driving the linear array transducer on the spindle to rotate, so as to obtain an ultrasound image at the corresponding longitudinal section.

The transducer assembly comprises at least two linear array transducers, which are spaced apart along the circumferential direction of the spindle. Each linear array transducer comprises multiple array elements linearly arranged along a preset direction of the spindle and can independently transmit ultrasound waves and receive returned ultrasound echoes. This allows the at least two linear array transducers, when excited, to transmit ultrasound waves in a radial direction of the spindle into a first target region within the pelvic cavity and receive returned ultrasound echoes, thereby obtaining at least two groups of ultrasound echo data corresponding to the at least two linear array transducers. The at least two groups of ultrasound echo data can be processed to obtain ultrasound images in at least two different longitudinal sections. This way, not only the scanning range of the intracavitary ultrasound probe in the pelvic cavity is increased to improve the comprehensiveness of the scanning of the intracavitary ultrasound probe, but also the examination efficiency of the intracavitary ultrasound probe in the pelvic cavity is improved, so as to shorten the time for the intracavitary ultrasound probe to perform annular scanning within the pelvic cavity and avoid shaking caused by prolonged holding during scanning so as to make the ultrasound images clearer. Furthermore, the appropriate ultrasound image can be selected as needed, reducing the workload of medical staff.

It should be understood that the general description above and the detailed description below are merely exemplary and explanatory, which does not limit the present disclosure.

### BRIEF DESCRIPTION OF THE DRAWINGS

To more clearly illustrate the technical solutions of the embodiments of the present disclosure, the drawings used in the description of the embodiments will be briefly introduced below. Obviously, the drawings described below are some embodiments of the present disclosure. For those skilled in the art, other drawings can be obtained based on these drawings without creative effort.

FIG. 1 is a schematic structural diagram of an ultrasound probe according to an embodiment of the present disclosure;

FIG. 2 is a cross-sectional schematic diagram of the ultrasound probe in FIG. 1;

FIG. 3 is a schematic exploded diagram of the ultrasound probe in FIG. 1;

FIG. 4 is a partial exploded diagram of the ultrasound probe in FIG. 1;

FIG. 5 is a schematic partial diagram of the ultrasound probe in FIG. 1;

FIG. 6 is a partial cross-sectional diagram of the ultrasound probe in FIG. 1;

FIG. 7 is a schematic partial diagram of the transducer assembly in FIG. 1;

FIG. 8 is a schematic structural diagram of the limiting assembly in FIG. 1;

FIG. 9 is a schematic cross-sectional diagram of the limiting assembly in FIG. 1;

FIG. 10 is an exploded diagram of the limiting assembly in FIG. 1;

FIG. 11 is another schematic diagram of the limiting assembly in FIG. 1;

FIG. 12 is a cross-sectional diagram of the limiting assembly in FIG. 11;

FIG. 13 is a schematic diagram of the control assembly and the driving assembly in FIG. 1;

FIG. 14 is a schematic structural diagram of the first reducer in FIG. 1;

FIG. 15 is a cross-sectional diagram of the first reducer in FIG. 1;

FIG. 16 is an exploded diagram of the first reducer in FIG. 1;

FIG. 17 is a schematic partial diagram of the driving assembly in FIG. 1; and

FIG. 18 is an exploded diagram of the second reducer in FIG. 1.

### EXPLANATION OF REFERENCE NUMERALS

10: Housing assembly; 11: Handle housing; 12: Probe housing; 121: Connecting housing; 122: Acoustic window; 13: Connecting seat; 131: Spindle rotation hole;
20: Transducer assembly; 21: Linear array transducer; 211: First linear array transducer; 212: Second linear array transducer; 213: Circuit board; 22: Spindle; 222: First bearing mounting part; 223: Second bearing mounting part; 224: Hollow cavity; 23: Bearing; 231: First bearing; 232: Second bearing; 233: Third bearing; 24: Fastening nut;
30: Driving assembly; 31: Driving component; 311: Driving gear; 32: First reducer; 321: First double gear; 3211: First input gear; 3212: First output gear; 322: Second double gear; 3221: Second input gear; 3222: Second output gear; 323: First transmission gear; 3231: First gear; 3232: Second gear; 3233: Gear shaft; 324: Output gear; 3241: Spindle fixing hole; 325: Mounting bracket; 3251: First receiving cavity; 3252: Second receiving cavity; 3253: First through hole; 3254: First shaft hole; 33: Encoder; 34: Second reducer; 342: Third double gear; 343: Fourth double gear; 344: Power output part; 312: Drive gear;
40: Limiting assembly; 41: Limiting component; 41a: Third groove; 411: First blocking part; 412: Limiting component mounting part; 42: Fixing component; 42a: Fourth groove; 421: Second blocking part; 422: First connecting platform; 43: Connecting component; 43a: First groove; 43b: Second groove; 431: First connecting component; 4311: First limiting part; 4311a: First upper limiting part; 4311b: First lower limiting part; 432: Second connecting component; 4321: Second limiting part; 4321b: Second upper limiting part; 4321b: Second lower limiting part; 4322: Second connecting platform; 44: First limiting component; 45: Second limiting component;
50: Control assembly; 51: Control board; 52: Fixing bracket;
60: Conductive layer.

### DETAILED DESCRIPTION

The technical solutions of the embodiments of the present disclosure will be clearly and completely described below with reference to the accompanying drawings. Obviously, the described embodiments are merely a part of the embodiments of the present disclosure, but not all of them. All other embodiments obtained by those skilled in the art based on the embodiments of the present disclosure without creative effort are within the scope of protection of the present disclosure.

It should also be understood that the terminology used in the present disclosure is merely for describing specific implements. It should be understood that, in the present disclosure, the orientations or positional relationships indicated by the terms such as “center”, “longitudinal”, “lateral”, “length”, “width”, “thickness”, “upper”, “lower”, “front”, “rear”, “left”, “right”, “vertical”, “horizontal”, “top”, “bottom”, “inner”, “outer”, “clockwise” and “counterclockwise”, etc. are based on the orientations or positional relationships shown in the accompanying drawings, which are merely for the convenience of describing the present disclosure and simplifying the description, but not indicate or imply that the related devices or components must have a specific orientation or be constructed and operated in a specific orientation. Therefore, they should not be construed as limitations to the present disclosure. Furthermore, the terms “first” and “second” are merely used for descriptive purposes and should not be construed as indicating or implying relative importance or implicitly specifying the number of indicated technical features. Therefore, the features specified as “first” or “second” may explicitly or implicitly comprise one or more of the specified features. In the description of the present disclosure, “multiple” means two or more, unless otherwise explicitly specified.

Some embodiments of the present disclosure will be described in details below in conjunction with the accompanying drawings. Unless otherwise specified, the following embodiments and the features in the embodiments can be combined with each other.

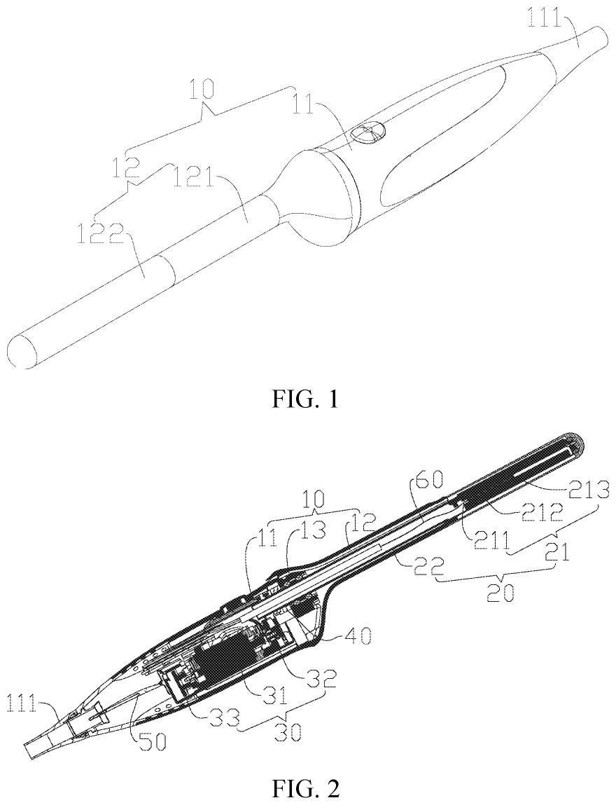

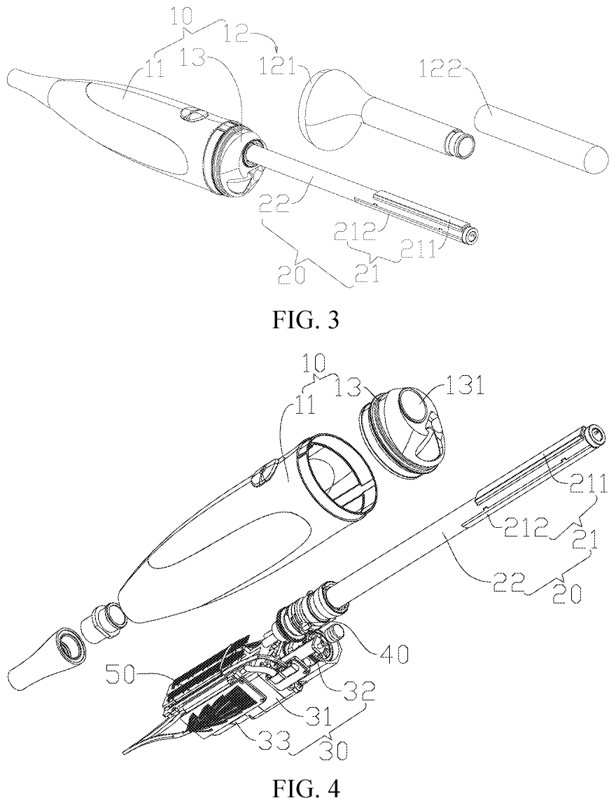

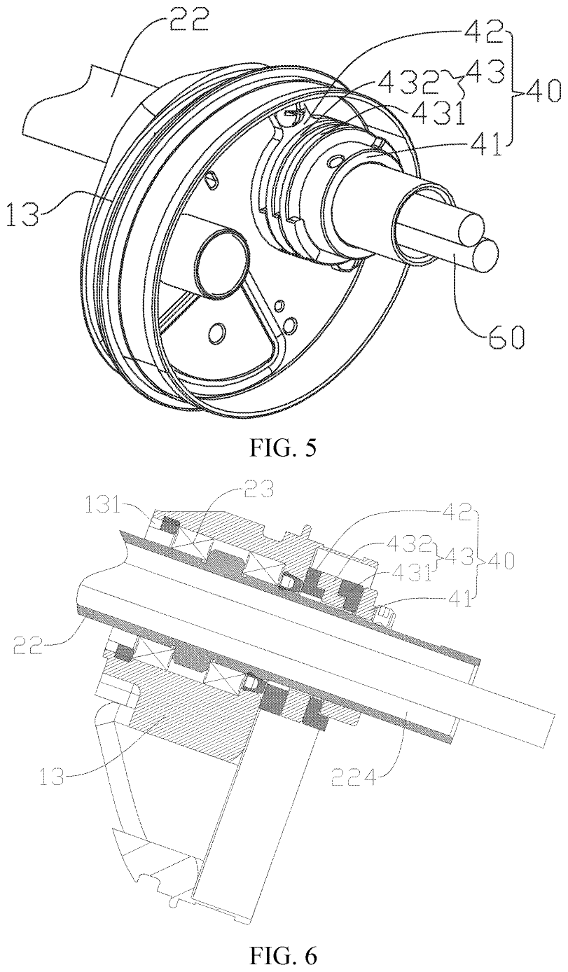

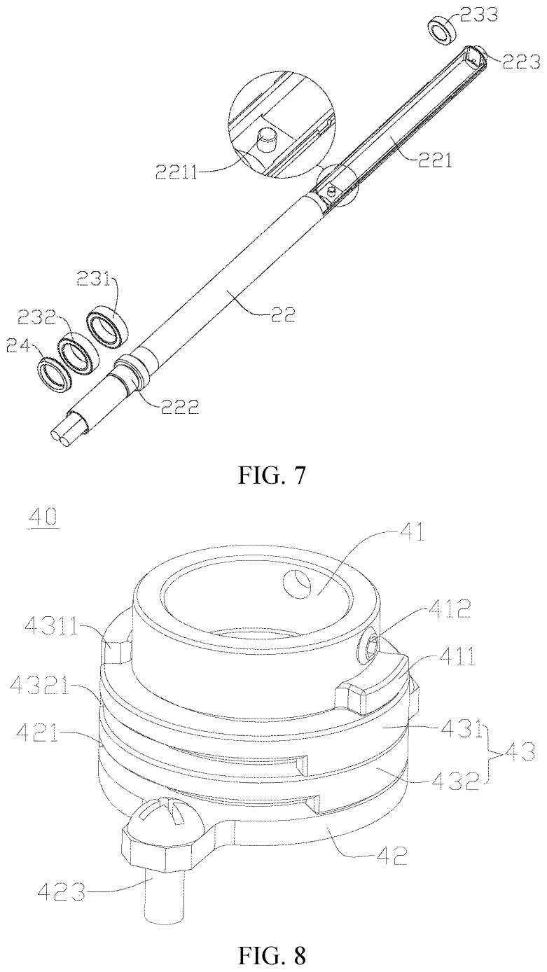

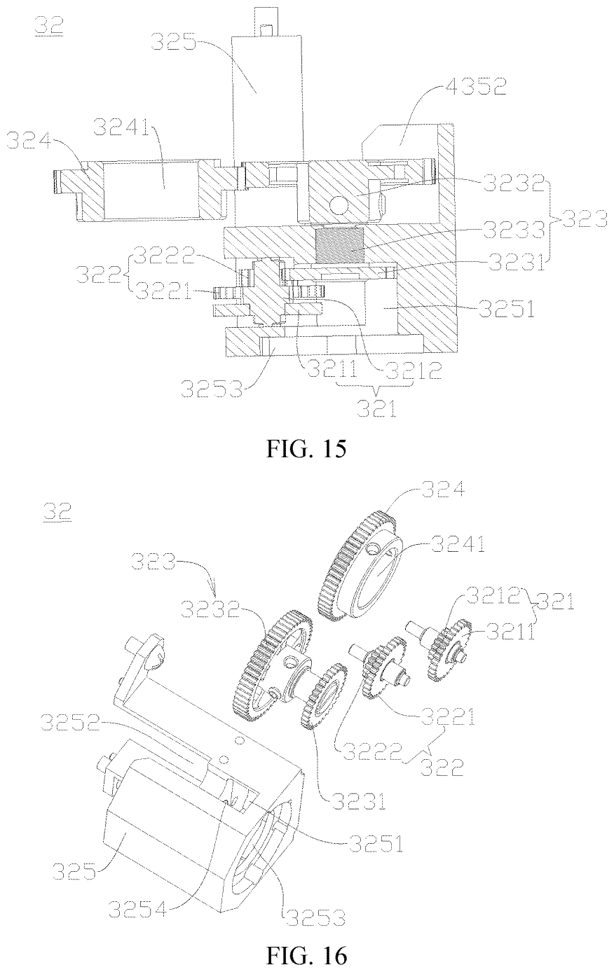

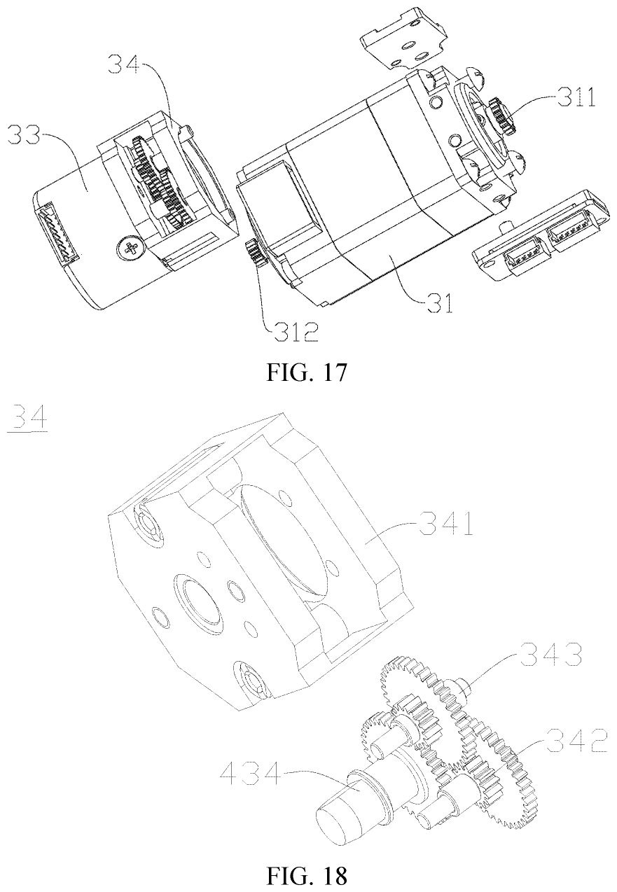

As shown in FIGS. 1 to 18, an intracavitary ultrasound probe for pelvic examination is provided, which may comprise a housing assembly 10, a driving assembly 30 and a transducer assembly 20 having a spindle 22. The housing assembly 10 may comprise a handle housing 11 and a probe housing 12 connected to the handle housing 11. The transducer assembly 20 may be disposed in the probe housing 12. The driving assembly 30 may be disposed in the handle housing 11, and be connected to the spindle 22 to drive the spindle 22 to rotate, so as to drive the linear array transducers 21 on the transducer assembly 20 to rotate around the central axis of the spindle 22 such that the rotational force generated by the driving assembly 30 is applied to the linear array transducers 21 of the transducer assembly 20 via the spindle 22, thereby rotatably driving the linear array transducers 21 to perform radial scanning operations to obtain corresponding ultrasound images.

In an embodiment, as shown in FIGS. 2 to 4, the transducer assembly 20 may comprise at least two linear array transducers 21. Each linear array transducer 21 may comprise multiple array elements arranged linearly along a preset direction of the spindle 22, and each array element is capable of independently transmitting and receiving ultrasound waves. The probe housing 12 may be provided with an acoustic window 122 and a connecting housing 121. The end of the connecting housing 121 facing away from the acoustic window 122 may be connected to the handle housing 11. The at least two linear array transducers 21 may be spaced apart in the circumferential direction of the spindle 22 and arranged in the acoustic window 122, allowing the spindle 22 to drive the at least two linear array transducers 21 to rotate around the central axis of the spindle 22. This enables each linear array transducer 21 to scan the longitudinal section of the pelvic cavity during rotation, thereby acquiring real-time ultrasound images in at least two different longitudinal sections when the intracavitary ultrasound probe scans the pelvic cavity.

It should be noted that the at least two linear array transducers 21 being spaced apart in the circumferential direction of the spindle 22 herein may comprise, but not limited to, the at least two linear array transducers 21 being partially overlapped in the radial direction of the spindle 22, or the at least two linear array transducers 21 being staggered from each other in the extension direction of the spindle 22, which is not limited in the present disclosure.

For example, after being excited, the at least two linear array transducers 21 can transmit ultrasound waves in the radial direction of the spindle 22 into a first target region in the pelvic cavity and receive the returned ultrasound echoes, thereby obtaining at least two groups of ultrasound echo data corresponding to the at least two linear array transducers 21. By processing these at least two groups of ultrasound echo data, ultrasound images in at least two different longitudinal sections may be obtained. This way, not only the scanning range of the intracavitary ultrasound probe in the pelvic cavity is increased to improve the comprehensiveness of the scanning of the intracavitary ultrasound probe, but also the examination efficiency of the intracavitary ultrasound probe in the pelvic cavity is improved, so as to shorten the time for the intracavitary ultrasound probe to perform annular scanning within the pelvic cavity and avoid shaking caused by prolonged holding during scanning so as to make the ultrasound images clearer. Furthermore, the appropriate ultrasound image can be selected as needed, reducing the workload of medical staff.

In the technical solution above, at least two linear array transducers 21 are provided on the spindle 22, each linear array transducer 21 is provided with array elements linearly arranged in a preset direction of the spindle 22, and each array element can independently transmit and receive ultrasound waves. Therefore, after each linear array transducer 21 is excited, each array element on each linear array transducer 21 can transmit ultrasound waves in the radial direction of the spindle 22 into the first target region in the pelvic cavity and receive the returned ultrasound echoes, thereby obtaining linear array ultrasound echo data corresponding to that linear array transducer 21. By processing the linear array ultrasound echo data, the ultrasound images in corresponding longitudinal sections can be obtained. The at least two linear array transducers 21 can obtain ultrasound images in at least two different longitudinal sections.

When the driving assembly 30 drives the spindle 22 to rotate around its central axis, the spindle 22 may drive the at least two linear array transducers 21 to rotate around the central axis of the spindle 22. This allows each linear array transducer 21 to acquire a series of ultrasound images during its rotation around the central axis of the spindle 22. This not only allows the ultrasound images of all linear array transducers 21 to be combined to form a composite image, such as a three-dimensional image, thereby shortening the scanning time of the intracavitary ultrasound probe in the pelvic cavity, but also avoids shaking caused by prolonged holding of the intracavitary ultrasound probe during the scanning process, resulting in clearer ultrasound images. Moreover, this also allows simultaneously obtaining the ultrasound images of the first target region at different tissue depths through the linear array transducers 21 with different operating frequency ranges without the movement or switching of the intracavitary ultrasound probe. Therefore, a single intracavitary ultrasound probe can achieve the scanning of the first target region at different depths. That is, multiple linear array transducers 21 with different operating frequency ranges on the spindle 22 may collaboratively transmit ultrasound waves into the first target region in the pelvic cavity and receive the returned echoes to obtain at least two groups of ultrasound echo data in at least two different longitudinal sections at different tissue depths, which significantly reduces the scanning time of the intracavitary ultrasound probe, greatly improves the efficiency of the intracavitary ultrasound probe, and avoids shaking caused by the prolonged holding of the intracavitary ultrasound probe during the scanning at different tissue depths, resulting in clearer ultrasound images. Furthermore, medical staff can select the appropriate ultrasound image as needed during the scanning of the intracavitary ultrasound probe, which reduces their workload.

In an embodiment, there may be two linear array transducers 21 that may be symmetrically arranged on both sides of the spindle 22 in a plane passing through the central axis of the spindle 22. The spindle 22 may comprise a front end and a rear end extending along the axial direction of the spindle 22. The driving assembly 30 may be connected to the rear end of the spindle 22 or to a position of the spindle 22 near the rear end. The two linear array transducers 21 may be arranged at the front end of the spindle 22 or at positions of the spindle 22 near the front end. When the driving assembly 30 drives the spindle 22 to rotate around its central axis, the spindle 22 can drive the two linear array transducers 21 to rotate simultaneously. After being excited, the two linear array transducers 21 can collaboratively transmit ultrasound waves in the radial direction of the spindle 22 into the first target region in the pelvic cavity and receive the returned ultrasound echoes to obtain two groups of ultrasound echo data corresponding to the two linear array transducers 21. By processing the two groups of ultrasound echo data, the real-time ultrasound images in corresponding two different longitudinal sections can be obtained. After the driving assembly 30 drives the spindle 22 to rotate one revolution around the central axis, each linear array transducer 21 can obtain a series of corresponding ultrasound images. If the operating frequency ranges of the two linear array transducers 21 are the same, the ultrasound images obtained by the two linear array transducers 21 can be combined to form a composite image, such as a three-dimensional image. Since the scanning arc length of the intracavitary ultrasound probe in the circumferential direction is equally divided by the two linear array transducers 21, the scanning path of the intracavitary ultrasound probe can be reduced by half, and therefore, the scanning time of the intracavitary ultrasound probe can also be reduced by half. If the operating frequency ranges of the two linear array transducers 21 are different, different penetration depth requirements can be met, allowing medical staff to quickly switch according to needs, or to perform the scanning to the tissues at different depths in two different longitudinal sections at one time to obtain ultrasound images of the tissues at different depths without moving or switching the ultrasound probe, which greatly improves the efficiency of intracavitary the ultrasound probe.

Similarly, when there are n linear array transducers 21, where n is an integer greater than 2, the n linear array transducers 21 may be spaced apart in the circumferential direction of the spindle 22 and arranged at the front end of the spindle 22 or at positions of the spindle 22 near the front end. When the driving assembly 30 drives the spindle 22 to rotate around its central axis, the n linear array transducers 21 can rotate simultaneously with the spindle 22 and collaboratively transmit ultrasound waves into the first target region in the pelvic cavity, and receive the returned ultrasound echoes to obtain n groups of ultrasound echo data corresponding to the n linear array transducers 21. By processing the n groups of ultrasound echo data, the real-time ultrasound images at n different longitudinal sections can be obtained. If the operating frequency ranges of the n linear array transducers 21 are the same, compared to the case where only one linear array transducer 21 is set on the spindle 22, the scanning path can be reduced to 1/n of the scanning path in said case, and the scanning time is also reduced to 1/n of the scanning time in said case. If the operating frequency ranges of the n linear array transducers 21 are at least partially different, it can not only shorten the scanning path and the scanning time, but also meet different penetration depth requirements, greatly improving the efficiency of the intracavitary ultrasound probe.

In an embodiment, the preset direction may be the axial direction of the spindle 22. Due to machining or assembly errors, slight deviations in the linear arrangement of multiple array elements in the preset direction of the spindle 22 are permissible, which will not be limited by the present disclosure.

In an embodiment, the transducer assembly 20 may comprise a first linear array transducer 211 and a second linear array transducer 212. The operating frequency range of the first linear array transducer 211 is the same as that of the second linear array transducer 212. This allows the first and second linear array transducers to work together to form a composite image, reducing the scanning path of the transducer assembly 20 and shortening the scanning time, and avoiding shaking caused by prolonged holding of the intracavitary ultrasound probe during scanning so as to obtain clearer ultrasound images. Furthermore, it allows for the selection of appropriate ultrasound images as needed, reducing the workload of medical staff.

In an embodiment, the transducer assembly 20 may comprise a first linear array transducer 211 and a second linear array transducer 212. The operating frequency range of the first linear array transducer 211 is different from that of the second linear array transducer 212, such that the ultrasound images of the tissues at different depths can be obtained during a single rotation without moving or switching the intracavitary ultrasound probe, greatly improving the examination efficiency of the intracavitary ultrasound probe. Alternatively, the first linear array transducer 211 or the second linear array transducer 212 at different frequencies can be quickly switched as needed to scan tissues at different depths.

It should be noted that the first linear array transducer 211 and the second linear array transducer 212 can be excited simultaneously during rotation around the central axis of the spindle 22. Alternatively, the first linear array transducer 211 and the second linear array transducer 212 can be excited individually during rotation around the central axis of the spindle 22. It can be performed as needed, and will not be limited by the present disclosure.

In an embodiment, the first linear array transducer 211 has the same dimensions as the second linear array transducer 212. That is, linear array transducers 21 of the same dimensions can be arranged on the spindle 22 as needed.

In an embodiment, the first linear array transducer 211 and the second linear array transducer 212 have different dimensions. That is, linear array transducers 21 of different dimensions can be arranged on the spindle 22 as needed.

In an embodiment, the distance from the front end of the first linear array transducer 211 to the front end of the spindle 22 is the same as the distance from the front end of the second linear array transducer 212 to the front end of the spindle 22. That is, at least the positions of the front ends of the first and second linear array transducers 211 and 212 on the spindle 22 are the same. In the case that the dimensions of the first and second linear array transducers 211 and 212 are the same, the installation positions of the first and second linear array transducers 211 and 212 on the spindle 22 are the same. In the case that the dimensions of the first and second linear array transducers 211 and 212 are different, the positions of the rear end of the first and second linear array transducers 211 and 212 on the spindle 22 are different. The specific configuration can be determined according to design and usage requirements, which will not be limited by the present disclosure.

In an embodiment, the distance from the front end of the first linear array transducer 211 to the front end of the spindle 22 may be different from the distance from the front end of the second linear array transducer 212 to the front end of the spindle 22. That is, the front ends of the first linear array transducer 211 and the second linear array transducer 212 on the spindle 22 can be staggered. The specific configuration can be determined according to design and usage requirements, which will not be limited by the present disclosure.

In an embodiment, as shown in FIGS. 2 to 4, the transducer assembly 20 may comprise only a first linear array transducer 211 and a second linear array transducer 212. The first linear array transducer 211 and the second linear array transducer 212 are symmetrically arranged on the two side walls of the spindle 22 with respect to the plane passing through the central axis of the spindle 22, such that the first linear array transducer 211 and the second linear array transducer 212 can transmit ultrasound waves in the radial direction of the spindle 22 to two longitudinal sections with opposite orientations, and receive the returned ultrasound echoes to obtain two groups of ultrasound echo data in the two longitudinal sections with opposite orientations. By processing the two groups of ultrasound echo data, the ultrasound images in the two longitudinal sections with opposite orientations can be obtained. After the first linear array transducer 211 and the second linear array transducer 212 rotate one revolution around the central axis of the spindle 22, both the first linear array transducer 211 and the second linear array transducer 212 acquire a series of ultrasound images. These ultrasound images can not only be used together for 3D reconstruction, but also be selected as needed, which reduces the workload of medical staff. Furthermore, they can be used to scan tissues at different depths to obtain ultrasound images of the corresponding depths without moving or switching the intracavitary ultrasound probe, which greatly improves the efficiency of the intracavitary ultrasound probe.

In an embodiment, the intracavitary ultrasound probe may further comprise a convex array transducer located at the front end of the spindle 22. The convex array transducer may be configured to transmit ultrasound waves into a second target region in the pelvic cavity and receive the returned ultrasound echoes to obtain a group of convex array ultrasound echo data corresponding to the convex array transducer. By processing the convex array ultrasound echo data, the ultrasound image in the corresponding transverse section can be obtained. That is, the convex array transducer can acquire the ultrasound echo data from the region near the front end of the intracavitary ultrasound probe to generate the ultrasound images of that region, thereby improving the examination angle range of the intracavitary ultrasound probe and resulting in better examination effects.

In an embodiment, the linear array transducer 21 may be rotated around the central axis of the spindle 22 and transmit and receive ultrasound waves in the radial direction of the spindle 22, thereby obtaining the linear array ultrasound echo data corresponding to the linear array transducer 21. By processing the linear array ultrasound echo data, the ultrasound images in the corresponding longitudinal section can be obtained. The convex array transducer, on the other hand, can transmit and receive ultrasound waves towards and from the region in front of the front end of the spindle 22 to obtain convex array ultrasound echo data corresponding to the convex array transducer. By processing the convex array ultrasound echo data, the ultrasound images in the corresponding transverse section can be obtained. When the linear array transducer 21 and the convex array transducer work simultaneously, the scanning of different regions can be achieved, and the composite image can also be generated, thereby improving the examination range of the intracavitary ultrasound probe.

It should be noted that the convex array transducer may also be rotated by the spindle 22 to scan the different sections in the region in front of the front end of the intracavitary ultrasound probe, which further improves the examination range. Meanwhile, the convex array transducer and the linear array transducer 21 may also work independently and scan two different regions separately, which will not be limited in the present disclosure.

In an embodiment, the end of the acoustic window 122 may be provided with an opening, and the convex array transducer may be arranged in the opening and sealed, such that the outer surface of the convex array transducer is flush with the outer surface of the acoustic window 122. The joint between the convex array transducer and the acoustic window 122 may be sealed.

It should be noted that when the intracavitary ultrasound probe is used to examine the pelvic cavity, the spindle 22 will be inserted into the pelvic cavity. Therefore, in order to improve the comfort of the examination and reduce patient discomfort, the body of the spindle 22 may be designed as cylindrical shape, and the front end of the spindle 22 may be designed as an arc shape that has a smooth transition with the body of the spindle 22. That is, the outer surface of the spindle 22 does not have sharp edges or other sharp structures, resulting in higher patient comfort during insertion.

In an embodiment, the convex array transducer may be mounted on the spindle 22 and be rotated about the central axis of the spindle 22. This allows the convex array transducer to scan different sections in the region in front of the front end of the probe when the spindle 22 rotates, increasing the examination range of the convex array transducer. There may be merely one driving assembly 30 to drive the convex array transducer and the linear array transducer 21 on the spindle 22 to rotate, which can simplify the structure and control strategy, facilitate the production and assembly, and control the costs.

In an embodiment, the intracavitary ultrasound probe may further comprise a rotating shaft that can rotate independently relative to the spindle 22. The convex array transducer may be connected to the rotating shaft. This allows the convex array transducer to rotate relative to the spindle 22. Therefore, the convex array transducer and the linear array transducer 21 need not to be rotated simultaneously, but can rotate independently as needed to expand its examination angle.

In an embodiment, the transducer assembly 20 may comprise a hinge shaft through which the convex array transducer is hinged to the front end and is capable of oscillating about the hinge shaft. The axis of the hinge shaft may be perpendicular to the axis of the spindle 22, allowing the convex array transducer to oscillate about the hinge shaft. This means that the convex array transducer can be driven to oscillate about the hinge shaft in a suitable manner, thereby improving the examination range of the convex array transducer.

In an embodiment, the convex array transducer may oscillate around the hinge axis at an angle greater than 0° and less than or equal to 180°. After the convex array transducer is excited, it can transmit ultrasound waves in the radial direction of the hinge axis towards the second target region and receive the returned ultrasound echoes to obtain the convex array ultrasound echo data corresponding to the convex array transducer. By processing the convex array ultrasound echo data, the ultrasound images of the tissue of the second target region in the transverse section can be obtained, which further improves the examination range of the intracavitary ultrasound probe and reduces the number of scans that the intracavitary ultrasound probe need to perform in the pelvic cavity without the needs to move or switch the intracavitary ultrasound probe. Therefore, the work efficiency is improved, the examination error caused by moving or switching the intracavitary ultrasound probe is reduced, and the examination results are more accurate.

In an embodiment, as shown in FIGS. 4 to 9, the housing assembly 10 may further comprise a connecting seat 13 with a spindle rotation hole 131. The connecting seat 13 may be connected between the handle housing 11 and the probe housing 12. One end of the spindle 22 may be rotatably mounted in the spindle rotation hole 131 and be connected to the driving assembly 30. The other end of the spindle 22 may extend into the acoustic window 122. Both the linear array transducer 21 and the convex array transducer may be disposed in the acoustic window 122 such that the ultrasound beams transmitted by the linear array transducer 21 and the convex array transducer can be focused. The acoustic window 122 can be made of various materials suitable for making acoustic lenses, such as polyethylene synthetic resin, etc.

In an embodiment, as shown in FIGS. 5 to 7, the transducer assembly 20 may further comprise a bearing 23 disposed in the spindle rotation hole 131 and the acoustic window 122. The spindle 22 may pass through the bearing 23 to facilitate smooth rotation of the spindle 22.

In an embodiment, the transducer assembly 20 may further comprise a fastening nut 24. The bearing 23 may comprise a first bearing 231, a second bearing 232 and a third bearing 233. The spindle 22 is provided with a first bearing mounting part 222 and a second bearing mounting part 223. The inner rings of the first bearing 231 and the second bearing 232 may be connected to the first bearing mounting part 222, and the outer rings of the first bearing 231 and the second bearing 232 may be connected to the inner wall of the spindle rotation hole 131. The fastening nut 24 may be locked to the outside of the second bearing 232 to fix the second bearing 232 to the spindle 22. The inner ring of the third bearing 233 may be connected to the second bearing mounting part 223, and the outer ring of the third bearing 233 may be fixed in the acoustic window 122.

It should be noted that the connecting seat 13 may be integrally formed with the handle housing 11 or the probe housing 12. Alternatively, the connecting seat 13 may be separately formed from the handle housing 11 or the probe housing 12 and then be fixed together by assembling. It will not be limited by the present disclosure.

In an embodiment, as shown in FIGS. 5 to 12, the intracavitary ultrasound probe may further comprise a limiting assembly 40. The limiting assembly 40 may be mounted on at least one of the handle housing 11, the probe housing 12, the connecting seat 13 and the spindle 22, allowing the spindle 22 to rotate at any angle within a range of not less than 720 degrees.

In an embodiment, the limiting assembly 40 may comprise a fixing component 42, a limiting component 41 and at least one connecting component 43. The fixing component 42 may be fixed to the housing assembly 10. The limiting component 41 may be fixed to the spindle 22. The connecting component 43 may be rotatably mounted on the spindle 22 and be disposed between the fixing component 42 and the limiting component 41 so as to form a limiting fit with the fixing component 42 and the limiting component 41 to restrict the spindle 22 to rotate within a stroke angle of not less than 720 degrees. This allows for limiting at any stroke angle, enabling adjustment of the angular position when the driving assembly 30 drives the transducer assembly 20 to rotate. This solves the technical problem of the transducer assembly 20 rotating at angles exceeding 720 degrees. Furthermore, it protects the electrical connection components inside the spindle 22 during torsion, preventing the electrical connection components like flexible circuit boards from being damaged during the rotation of the spindle, thereby improving the service life of the electrical connection components.

It should be noted that the transducer assembly 20's rotation within a stroke angle of not less than 720 degrees may mean that the transducer assembly 20 can rotate more than two revolutions or equal to two revolutions around its central axis in either a counterclockwise or clockwise direction, or may mean that the total stroke angle of the transducer assembly 20's rotation around its central axis in both counterclockwise and clockwise directions is not less than 720 degrees. It may be depended on the design requirements. For example, it could be rotation within 0 to 720 degrees, −360 to +360 degrees, or 0 to 810 degrees, or even rotation exceeding 810 degrees, etc. Here, 0 degrees may be the initial rotation angle of the transducer assembly 20, and the aforementioned 720 degrees or 810 degrees, etc. may be the final rotation angles of the transducer assembly 20 in one direction. The aforementioned −360 and +360 degrees may be the final rotation angles of the transducer assembly 20 in the counterclockwise direction and the final rotation angle in the clockwise direction, respectively. The initial rotation angle of the transducer assembly 20 may also be −180 degrees or −360 degrees, etc. Correspondingly, in this case, the final rotation angle of the transducer assembly 20 may be the difference between the stroke angle of the transducer assembly 20 and the initial rotation angle of the transducer assembly 20.

In an embodiment, the rotation of the spindle 22 within a stroke angle of not less than 720 degrees may comprise the rotation from a first stroke angle to a second stroke angle. The first stroke angle may be either the initial rotation angle or the reverse final rotation angle of the transducer assembly 20, and the second stroke angle may be the forward final rotation angle of the transducer assembly 20, where the forward rotation may be one of the either clockwise rotation and the counterclockwise rotation, and the reverse rotation may be the other of the clockwise rotation and the counterclockwise rotation.

In the technical solutions above, the transducer assembly 20 can rotate within a preset rotation angle between the first stroke angle and the second stroke angle under the cooperation of the fixing component 42, the limiting component 41 and the at least one connecting component 43. This not only prevents the transducer assembly 20 from exceeding its limit position during the rotation, thus avoiding damage to the internal electrical connection components, but also ensures that the rotation angle of the transducer assembly 20 is greater than 720 degrees, enabling imaging within the effective motion range of 720 degrees. Compared to the method that relies solely on a combination of motor pulse count and zero-return control, the present disclosure offers higher reliability. In particular, problems occurred in the program for motor pulse count and zero-return control may damage the internal electrical connection components of the transducer assembly 20, leading to transducer assembly 20 failure.

In an embodiment, when the first stroke angle is 0 degrees and the second stroke angle is 720 degrees, the driving assembly 30 can drive the spindle 22 to rotate between 0 degree and 720 degrees to protect the electrical connection components inside the spindle 22, thereby preventing the electrical connection components from affecting the transmission effect of the transducer assembly 20 when the electrical connection components are twisted or squeezed. Alternatively, when the first stroke angle is 0 degree and the second stroke angle is greater than 720 degrees, the driving assembly 30 can drive the spindle 22 to rotate between 0 degrees and any angle more than 720 degrees, so as to achieve a large angle selection of the transducer assembly 20, thereby meeting various angle requirements of the transducer assembly 20. This improves the scanning efficiency of the transducer assembly 20, and also protects the electrical connection components inside the spindle 22, preventing the electrical connection components from affecting the transmission effect of the transducer assembly 20 or even being damaged when the electrical connection components are twisted or squeezed. Alternatively, when the first stroke angle is −360 degrees and the second stroke angle is +360 degrees, the driving assembly 30 can drive the spindle 22 to rotate at any angle between −360 degrees and +360 degrees, so as to realize the free angle selection of the forward and reverse rotation of the transducer assembly 20, thereby meeting the various angle requirements of the transducer assembly 20.

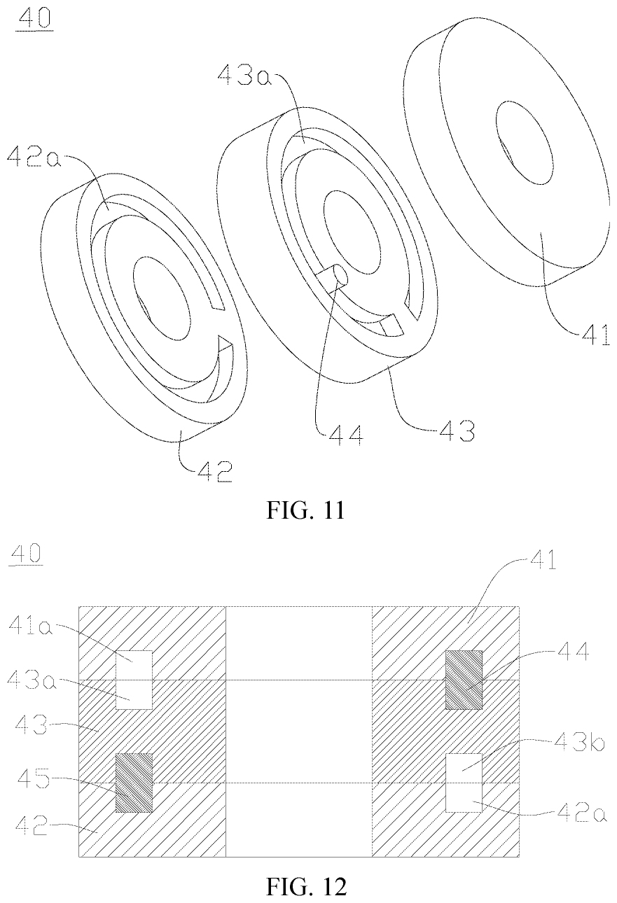

In an embodiment, as shown in FIGS. 11 and 12, the connecting component 43 may have a first end face and a second end face opposite to the first end face. A first limiting component 44 may be connected between the first end face and the limiting component 41, and a second limiting component 45 may be connected between the second end face and the fixing component 42. The first limiting component 44 and the second limiting component 45 may be rotated relative to the connecting component 43, the limiting component 41 and the fixing component 42 about the axis of the spindle 22. When the driving assembly 30 drives the spindle 22 to rotate, since the limiting component 41 is fixed on the spindle 22, the fixing component 42 is fixed on the housing assembly 10 and the connecting component 43 is sleeved on the spindle 22 and can be rotated relative to the spindle 22, the spindle 22 drives the limiting component 41 to rotate, and when the limiting component 41 is moved to the first limiting component 44 and the first limiting component 44 contacts the limiting end face of the limiting component 41, the limiting component 41 drives the first limiting component 44 to rotate around the axis of the spindle 22, and when the first limiting component 44 contacts the limiting end face of the connecting component 43 on the first end face, the limiting component 41 drives the connecting component 43 to rotate relative to the fixing component 42 through the first limiting component 44, and when the limiting end face of the connecting component 43 on the second end face contacts the second limiting component 45, the connecting component 43 drives the second limiting component 45 to rotate around the axis of the spindle 22 until the second limiting component 45 contacts the limiting end face of the fixing component 42. The movement of the second limiting component 45 is blocked by the limiting end face on the fixing component 42, which prevents the connecting component 43 from continuing to rotate relative to the fixing component 42, and further prevents the limiting components 41 from continuing to rotate through the connecting component 43 and the first limiting component 44, thereby achieving the mechanical limiting function. This solves the technical challenge of rotating the transducer assembly 20 more than 720 degrees, and also protects the electrical connection components inside the spindle 22 during the torsion process of the spindle 22. It can not only achieve the control of the pulse count of the driving assembly 30, but also achieve the mechanical limiting of the spindle 22 during the rotation through the limiting component 40. The structure is simple and the operation is reliable.

In an embodiment, a first groove 43a is provided on the first end face, a second groove 43b is provided on the second end face, a third groove 41a is provided on the limiting component 41, and a fourth groove 42a is provided on the fixing component 42. The first limiting component 44 is slidably mounted between the first groove 43a and the third groove 41a, and the second limiting component 45 is slidably mounted between the second groove 43b and the fourth groove 42a, such that the limiting component 41 and the connecting component 43 can be mechanically limited by the first limiting component 44 connected between the first groove 43a and the third groove 41a, and the connecting component 43 and the fixing component 42 can be mechanically limited by the second limiting component 45 connected between the second groove 43b and the fourth groove 42a. The limiting angle between the limiting component 41 and the connecting component 43 may be determined by the radian of the first groove 43a and the radian of the third groove 41a. The limiting angle between the connecting component 43 and the fixing component 42 may be determined by the radian of the second groove 43b and the radian of the fourth groove 42a. That is, the mechanical limiting angle of the limiting assembly 40 may be determined by the rotation angle of the connecting component 43 relative to the spindle 22 and the radian of the first groove 43a, the radian of the second groove 43b, the radian of the third groove 41a and the radian of the fourth groove 42a.

In an embodiment, the first groove 43a has two first limiting end faces that are opposite to each other, and the first limiting component 44 can rotate around the axis of the spindle 22 from one of the first limiting end faces to the other of the first limiting end faces. Similarly, the third groove 41a has two third limiting end faces that are opposite to each other, and the first limiting component 44 can rotate around the axis of the spindle 22 from one of the third limiting end faces to the other of the third limiting end faces. When the first limiting component 44 is jointly limited by the first limiting end face and the third limiting end face, the limiting component 41 can drive the connecting component 43 to rotate relative to the spindle 22 through the first limiting component 44. Similarly, the second groove 43b has two second limiting end faces that are opposite to each other, and the second limiting component 45 can rotate around the axis of the spindle 22 from one of the second limiting end faces to the other of the second limiting end faces. The fourth groove 42a has two fourth limiting end faces that are opposite to each other, and the second limiting component 45 can rotate around the axis of the spindle 22 from one of the fourth limiting end faces to the other of the fourth limiting end faces. When the limiting component 41 drives the connecting component 43 to rotate through the first limiting component 44, the second limiting component 45 can rotate in the second groove 43b around the axis of the spindle 22 until the second limiting component 45 is blocked by the second limiting end face, from which position the second limiting component 45 will be rotated around the axis of the spindle 22 along with the connecting component 43 until it is blocked by the fourth limiting end face. Since the fixing component 42 is fixed to the housing assembly 10, the fourth limiting end face is fixed relative to the housing assembly 10, thereby limiting the rotation angle of the second limiting component 45. The second limiting component 45 can then limit the rotation angle of the connecting component 43 via the second limiting end face, the connecting component 43 can limit the rotation angle of the first limiting component 44 via the first limiting end face, and the first limiting component 44 can then limit the rotation angle of the limiting component 41 via the third limiting end face.

In an embodiment, as shown in FIGS. 5 to 10, the connecting component 43 may comprise a first connecting component 431 and a second connecting component 432 that forms a limiting fit with the first connecting component 431. The first connecting component 431 forms a limiting fit with the limiting component 41 to limit the rotation angle of the connecting component 43, and the second connecting component 432 forms a limiting fit with the fixing component 42 to limit the rotation angle of the connecting component 43, such that the spindle 22 achieves three-level mechanical limiting through the limiting fit between the limiting component 41 and the first connecting component 431, the limiting fit between the first connecting component 431 and the second connecting component 432, and the limiting fit between the second connecting component 432 and the fixing component 42. This solves the technical challenge of rotating the transducer assembly 20 more than 720 degrees, and also protects the electrical connection components inside the spindle 22 during the torsion process of the spindle 22. It can not only achieve the control of the pulse count of the driving assembly 30, but also achieve the mechanical limiting of the spindle 22 during the rotation through the limiting component 40. The structure is simple and the operation is reliable.

It should be noted that the relative rotation angle between the limiting component 41 and the first connecting component 431 may be between 0 degrees and 360 degrees, such as 240 degrees. The relative rotation angle between the first connecting component 431 and the second connecting component 432 may also be between 0 degrees and 360 degrees, such as 240 degrees. Similarly, the relative rotation angle between the second connecting component 432 and the fixing component 42 may also be between 0 degrees and 360 degrees, such as 240 degrees. Therefore, the limiting angle of the entire limiting assembly 40 can be 720 degrees or even greater than 720 degrees, which will not be limited in the present disclosure.

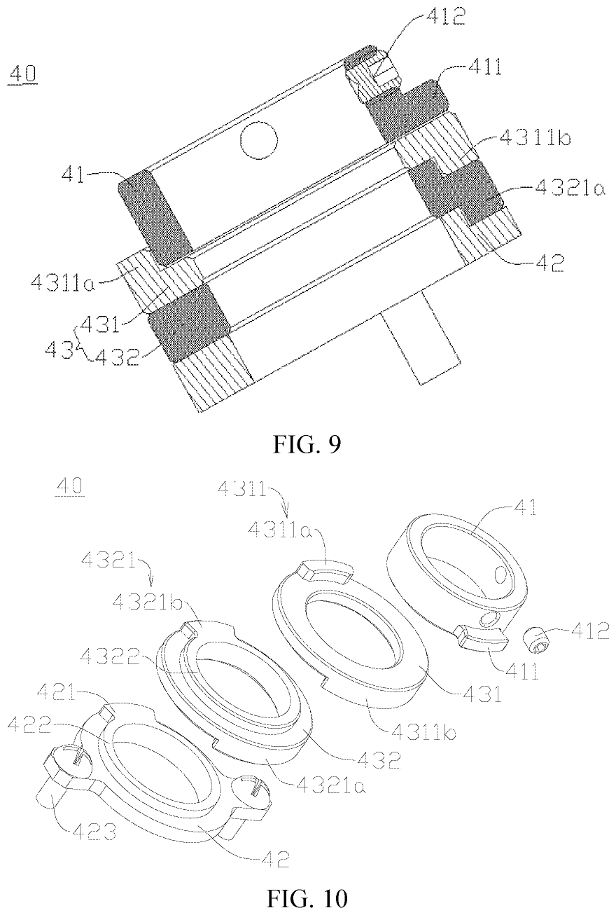

In an embodiment, as shown in FIGS. 9 and 10, the first connecting component 431 may be provided with a first limiting part 4311 at each of its opposite ends, and the second connecting component 432 may be provided with a second limiting part 4321 at each of its opposite ends. One of the first limiting part 4311 and one of the second limiting part 4321 form a limiting fit, the other of the first limiting part 4311 and the limiting component 41 form a limiting fit, and the other of the second limiting part 4321 and the fixing component 42 form a limiting fit. This way, the first connecting component 431 and the second connecting component 432 achieve jointly the limiting fit with the limiting part and the fixing component 42. It solves the technical challenge of rotating the transducer assembly 20 more than 720 degrees so as to meet various large-angle rotation needs for transducer assembly 20. Furthermore, it prevents damage to the electrical connection components inside the spindle 22 during the twisting process of the spindle 22.

In an embodiment, the first limiting part 4311 may comprise a first upper limiting part 4311a facing the limiting component 41 and a first lower limiting part 4311b facing the second connecting component 432, and the second limiting part 4321 may comprise a second upper limiting part 4321b facing the first connecting component 431 and a second lower limiting part 4321b facing the fixing component 42. The first upper limit portion 4311a may be used to form a limiting fit with the limiting component 41 to prevent the limiting component 41 from rotating or enable the limiting component 41 to drive the first connecting component 431 to rotate through the first upper limit portion 4311a. The first lower limit portion 4311b may be used to form a limiting fit with the second upper limit portion 4321b to prevent the first connecting component 431 from rotating or enable the first connecting component 431 to drive the second connecting component 432 to rotate through the fit between the first lower limit portion 4311b and the second upper limit portion 4321b. The second lower limit portion 4321b may be used to form a limiting fit with the fixing component 42 to prevent the second connecting component 432 from rotating.

In an embodiment, the limiting component 41 may be provided with a first blocking part 411 disposed on the outer periphery of the limiting component 41. The first blocking part 411 may be used to form a limiting fit with the first limiting part 4311 disposed on the limiting component 41, allowing the limiting component 41 to form a mechanical limit through the first blocking part 411 and the first limiting part 4311. Furthermore, the first blocking part 411's location on the outer periphery of the limiting component 41 facilitates its forming and processing.

In an embodiment, the inner surface of the first limiting part 4311 contacts the outer periphery of the limiting component 41, allowing the limiting component 41 to rotate against the inner surface of the first limiting part 4311 so as to improve the stability of the first connecting component 431 when rotating around the axis of the spindle 22.

In an embodiment, the fixing component 42 may be provided with a first connecting platform 422 and a second blocking part 421 disposed on one side of the first connecting platform 422. The inner surface of the second limiting part 4321 contacts the outer periphery of the first connecting platform 422 and is used to form a limiting fit with the second blocking part 421. Furthermore, the close contact between the second limiting part 4321 and the first connecting platform 422 can improve the stability of the second connecting component 432 during rotation.

In an embodiment, the second connecting component 432 may be provided with a second connecting platform 4322 on the side facing the first connecting component 431. The second limiting part 4321 is connected to the outer periphery of the second connecting platform 4322. The inner surface of the first limiting part 4311 contacts the outer periphery of the second connecting platform 4322, such that the first connecting component 431 can rotate against the outer periphery of the second connecting platform 4322 through the first limiting part 4311, and then form a limiting fit with the second limiting part 4321. This not only improves the stability between the first connecting component 431 and the second connecting component 432, but also facilitates the machining and forming of the second connecting platform 4322 and the second limiting part 4321.

In an embodiment, the limiting component 41 may be provided with a limiting component mounting part 412, which is used to fix the limiting component 41 to the spindle 22.

In an embodiment, the limiting component 41 may comprise a body and a fixing screw. The body is provided with a screw hole, and the fixing screw is threaded into the screw hole to achieve a fixed connection between the body and the spindle 22.

In an embodiment, the fixing component 42 may be mounted on the connecting seat 13, and the limiting component 41 may be fixed to the end of the spindle 22 extending out of the connecting seat 13.

In an embodiment, the driving assembly 30 may comprise a driving component 31 and a first reducer 32. The driving component 31 may be connected to the spindle 22 through the first reducer 32, such that the driving assembly 30 can reduce the output speed of the driving component 31 through the transmission gears in the first reducer 32, thereby increasing the rotational force transmitted by the driving component 31 to the spindle 22, which can significantly improve the control accuracy of the ultrasound transducer 21 and ensure that sufficiently torque is outputted to the ultrasound transducer 21 to overcome the resistance of the electrical connection components inside the spindle 22 during the torsion of the spindle 22 or the resistance applied to the transducer assembly 20 by the tissue inside the body cavity during the examination.

Specifically, during the examination, the ultrasound probe is inserted into the body cavity to perform a complete circumferential scan to acquire data for the examined area, such as when examining the pelvic cavity. When the ultrasound probe scans in the body cavity, the driving component 31 drives the spindle 22 to rotate in the cavity. The body cavity wall on the outer periphery of the spindle 22 generates a resistance force in the opposite direction to the spindle 22 to resist the rotation of the spindle 22. This resistance force may reduce the rotational speed of the spindle 22, or may even force the spindle 22 to stop rotating. Furthermore, in order to improve the control accuracy of the ultrasound probe, the rotational speed of the spindle 22 in the body cavity cannot be too fast. Due to the limitation of the internal space of the ultrasound probe, the output of the stepper motor is typically transmitted to the spindle 22 via a single-stage spur gear, which is insufficient to meet the driving force required by the spindle 22. Therefore, in the present disclosure, while meeting the dimensional requirements, the first reducer 32 is connected between the driving component 31 and the spindle 22, which can not only satisfy the driving force required by the spindle 22 through the principle of increasing torque through deceleration, but also achieve the normal acceleration, uniform speed and deceleration of the spindle 22 through the driving component 31, thereby significantly improving the control accuracy of the ultrasound transducer 21.

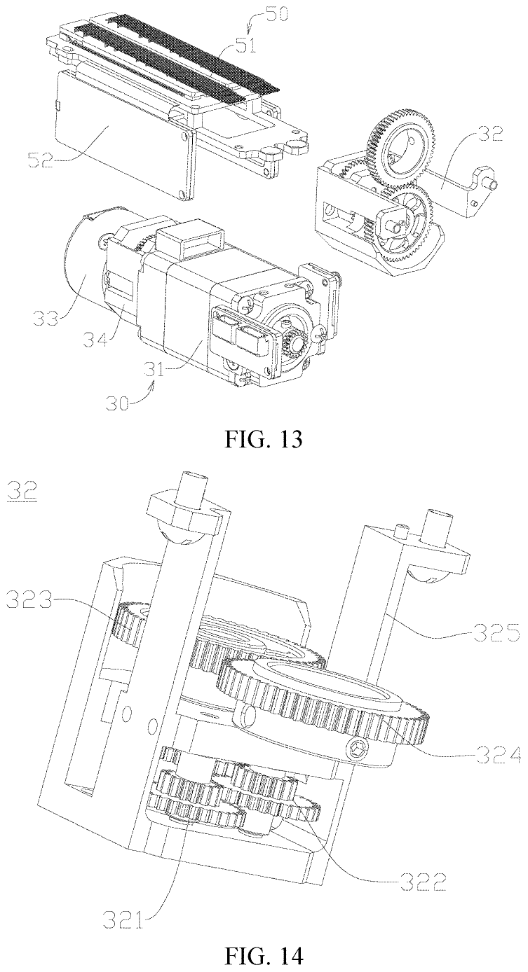

In an embodiment, as shown in FIGS. 13 to 16, the first reducer 32 may comprise a mounting bracket 325, an output gear 324 and multiple transmission gears disposed in the mounting bracket 325. The driving component 31 may comprise a motor and a driving gear 311 connected to the motor shaft. The spindle 22 is connected to the output gear 324. The multiple transmission gears mesh sequentially from the driving gear 311 to the output gear 324, allowing the driving component 31 to drive the output gear 324 to rotate the spindle 22 after the gradual deceleration through the transmission gears, thereby achieving increasing torque through deceleration. This not only provides the driving force required by the spindle 22, but also significantly improves the control accuracy of the ultrasound transducer 21.

When the ultrasound probe rotates in the body cavity, the cavity wall will apply resistance force to the ultrasound transducer 21 inserted in the cavity to resist the rotation of the spindle 22. If this resistance force exceeds the rotational resistance of the motor, the motor will stop working. Therefore, in the present disclosure, multiple transmission gears are used to progressively reduce the speed to increase the torque to the spindle 22, such that the torque transmitted from the motor to the spindle 22 through the first reducer 32 is much greater than the original output torque of the motor, thereby overcoming the resistance force of the cavity wall during the rotation of the spindle 22 and the resistance force generated by the electrical connection components inside the spindle 22 during the torsion.

It should be noted that the rotational speed of the spindle 22 can be controlled by the motor, but the original output torque of the motor is generally lower than the resistance force encountered by the spindle 22 during rotation in the body cavity. That is, the design torque or original output torque of the motor cannot meet the driving force required by the spindle 22.

In an embodiment, the mounting bracket 325 may comprise a frame and a partition disposed in the frame. The partition divides the frame into a first receiving cavity 3251 and a second receiving cavity 3252. The driving gear 311 meshes with the transmission gear arranged in the first receiving cavity 3251, and the output gear 324 meshes with the transmission gear arranged in the second receiving cavity 3252. While achieving the decelerate function of the first reducer 32, this design results in a more compact structure, a more rational layout, and an effective reduction in the volume of the first reducer 32, thereby saving the manufacturing costs.

In an embodiment, the transmission gear may comprise a first transmission gear 323, which comprises a first gear 3231, a gear shaft 3233 and a second gear 3232. A first shaft hole 3254 is provided on the partition plate, and the gear shaft 3233 is rotatably mounted in the first shaft hole 3254. The first gear 3231 and the second gear 3232 are respectively disposed at two ends of the gear shaft 3233 located in the first receiving cavity 3251 and the second receiving cavity 3252. The output gear 324 meshes with the second gear 3232, and the driving gear 311 is connected to the first gear 3231 to achieve a transmission connection between the transmission gears in the first receiving cavity 3251 and the second receiving cavity 3252. This allows the driving gear 311 to transmit the output torque of the motor to the gear shaft 3233 through the first gear 3231, and transmit the output torque of the gear shaft 3233 to the spindle 22 through the meshing between the second gear 3232 and the output gear 324, thereby driving the spindle 22 to rotate. In the present disclosure, the diameter of the second gear 3232 may be larger than the diameter of the first gear 3231.

In an embodiment, the transmission gears may further comprise a first double gear 321, which comprises a first input gear 3211 and a first output gear 3212. The first input gear 3211 meshes with the driving gear 311, and the first output gear 3212 meshes with the first gear 3231. The diameter of the first input gear 3211 is larger than the diameter of the first output gear 3212. This integrated double gear design can not only ensure that the first double gear 321 can increase the reduction ratio of the first reducer 32 within the allowable strength of the first reducer 32, thereby increasing the output torque of the first reducer 32, but also reduce the volume and weight of the first reducer 32, thereby making the first reducer 32 to be more compact and lowering the manufacturing cost of the first reducer 32.

In an embodiment, the transmission gear may further comprise a second double gear 322, which comprises a second input gear 3221 and a second output gear 3222. The second input gear 3221 meshes with the first output gear 3212, and the second output gear 3222 meshes with the first gear 3231. The diameter of the second input gear 3221 is larger than the diameter of the second output gear 3222. Using the double gear can not only make the first reducer 32 to be more compact and smaller, but also facilitate the installation of the first reducer 32 and reduce the manufacturing costs.

In an embodiment, both the first double gear 321 and the second double gear 322 may be disposed in the first receiving cavity 3251. The frame is provided with a first through hole 3253. The driving gear 311 passes through the first through hole 3253 and meshes with the first input gear 3211 to transmit the motor's output torque to the gear shaft 3233 and the first input gear 3211. Through the meshing of the first output gear 3212 with the second input gear 3221, the output torque on the first double gear 321 is transmitted to the second double gear 322. Through the meshing of the second output gear 3222 with the first gear 3231, the output torque on the second double gear 322 is transmitted to the first transmission gear 323. Through the meshing of the second gear 3232 with the output gear 324, the output torque on the first transmission gear 323 is transmitted to the spindle 22, thereby driving the ultrasound transducer 21 on the spindle 22 to rotate to perform the scanning operation in the body cavity to obtain corresponding ultrasound images.

In an embodiment, as shown in FIGS. 13, 17 and 18, the driving assembly 30 may further comprise an encoder 33, which may be disposed on the side of the motor opposite to the first reducer 32. The encoder 33 may be configured to detect the rotational speed of the spindle 22, thereby improving the control accuracy of the ultrasound probe.

In an embodiment, a second reducer 34 may be connected between the encoder 33 and the motor. The transmission ratio of the second reducer 34 may correspond to the transmission ratio of the first reducer 32, such that the encoder 33 can match the output speed of the spindle 22, thereby significantly improving the control accuracy of the ultrasound probe and making the motor operation more stable and reducing the vibration.

In an embodiment, the second reducer 34 may comprise a third double gear 342, a fourth double gear 343 and a power output part 344. The motor may be provided with a drive gear 312 at the end away from the first reducer 32. The drive gear 312 may be connected to the power output part 344 through the third double gear 342 and the fourth double gear 343. The power output part 344 may be connected to the encoder 33, such that the operation status of the motor can be transmitted to the control assembly 50 of the ultrasound probe in real time through the encoder 33. This allows the control assembly 50 to analyze the operation of the motor, record the speed and position of the motor, and make appropriate feedback, thereby maintaining the high accuracy of the motor at high or low torque.

In an embodiment, as shown in FIGS. 4, 6 and 16, one end of the spindle 22 may be rotatably mounted in the spindle rotation hole 131 and be connected to the output gear 324. The mounting bracket 325 may be fixed to the connecting seat 13. In this embodiment, the output gear 324 may be provided with a spindle fixing hole 3241. The inner diameter of the spindle fixing hole 3241 may match the outer diameter of the spindle 22, allowing the spindle 22 to be fixed in the spindle fixing hole 3241.

In an embodiment, the ultrasound probe may further comprise a control assembly 50 which may be disposed in the handle housing 11. The transducer assembly 20 and the driving assembly 30 are both electrically connected to the control assembly 50, enabling the control assembly 50 to control the operation of the driving assembly 30 and the transducer assembly 20.

In an embodiment, the control assembly 50 may comprise a control board 51 and a fixing bracket 52. The control board 51 may be fixed to the outside of the motor via the fixing bracket 52. The control board 51 may be connected to the transducer assembly 20 and the driving assembly 30 by cables so as to control the operations of the transducer assembly 20 and the driving assembly 30.

In an embodiment, a hollow cavity 224 may be formed in the spindle 22. A conductive layer 60 for signal transmission may be disposed in the hollow cavity 224. At least two linear array transducers 21 may be connected to the sidewall of the spindle 22 and be connected with the conductive layer 60. The linear array transducer 21 may comprise a support frame, an acoustic lens, a backing layer and a piezoelectric layer electrically connected to the conductive layer 60. The acoustic lens may be disposed outside the piezoelectric layer, and the piezoelectric layer may be disposed on the support frame via the backing layer. The support frame may be connected to the sidewall of the spindle 22.

In an embodiment, a circuit board 213 may connect the at least two ultrasound transducers 21. The circuit board 213 may be disposed on the side of the support frame opposite to the backing layer. The piezoelectric layer may be electrically connected to the circuit board 213, and the circuit board 213 may be connected to the conductive layer 60. The circuit board 213 may comprise, but not limited to, a flexible FPC board.

In an embodiment, a first connector may be disposed on the side of the support frame opposite to the backing layer, and a second connector may be disposed on the circuit board 213. The first connector maybe connected with the second connector to achieve quick insertion and electrical connection between the piezoelectric layer and the circuit board 213.

In an embodiment, an ultrasound imaging device may be provided. The ultrasound imaging device may comprise a display, an ultrasound host and the ultrasound probe described above. The ultrasound probe is connected to the ultrasound host. The ultrasound host may be configured to process the at least two groups of ultrasound echo data to obtain the ultrasound images in at least two different longitudinal sections. The display may be configured to display the obtained ultrasound images.

In the description of the present disclosure, it should be noted that, unless otherwise expressly specified and limited, the terms “installation”, “connection” and “linking” should be interpreted broadly. For example, they may refer to a fixed connection, a detachable connection, or an integral connection. They may be mechanical connections or electrical connections. They may be direct connections or indirect connections through an intermediate medium, and may refer to the internal communication of two components or the interaction between two components. Those skilled in the art can understand the specific meaning of the above terms in the present disclosure according to the specific circumstances.

In the present disclosure, unless otherwise expressly specified and limited, a first feature being “above” or “below” a second feature may mean a direct contact between the first and second features; or mean a contact between the first and second features through another feature between them, but not direct contact. Furthermore, a first feature being “above”, “over” or “on top” of a second feature may mean that the first feature directly above or diagonally above the second feature, or may simply mean that the first feature is at a higher horizontal level than the second feature. A first feature being “below”, “lower” or “under” of a second feature may mean that the first feature directly below or diagonally below the second feature, or may simply mean that the first feature is at a lower horizontal level than the second feature.

The description above provides many different embodiments or examples for implementing the present disclosure. To simplify the disclosure of the present disclosure, the components and arrangements of certain examples have been described above. These are merely examples and are not intended to limit the scope of the present disclosure. Furthermore, reference numerals and/or letters may be repeated in different examples. Such repetition is for simplification and clarity, but does not in itself indicate a relationship between the various embodiments and/or arrangements discussed. In addition, examples of various specific processes and materials have been provided in the present disclosure. However, those skilled in the art will recognize the application of other processes and/or the use of other materials.

In the description of this specification, the description with reference to “one embodiment”, “some embodiments”, “illustrative embodiment”, “an example”, “a specific example” or “some examples”, etc. may mean that the specific features, structures, materials or characteristics described in connection with the embodiments or examples are comprised in at least one embodiment or example of the present disclosure. In this specification, the illustrative expressions to the terms above do not necessarily refer to the same embodiments or examples. Moreover, the specific features, structures, materials or characteristics described may be combined in any suitable manner in one or more embodiments or examples.

What is claimed is:

1. An intracavitary ultrasound probe for pelvic examination, comprising a housing assembly, a driving assembly and a transducer assembly having a spindle, the housing assembly comprising a handle housing and a probe housing connected to the handle housing, the transducer assembly being arranged in the probe housing, and the driving assembly being arranged in the handle housing and connected to the spindle to drive the spindle to rotate about a central axis of the spindle;
wherein, the transducer assembly comprises at least two linear array transducers, each of which comprises multiple array elements linearly arranged along a preset direction of the spindle, the probe housing is provided with an acoustic window and a connecting housing, an end of the connecting housing facing away from the acoustic window is connected to the handle housing, and the at least two linear array transducers are spaced apart in a circumferential direction of the spindle and arranged in the acoustic window such that the spindle is able to drive the at least two linear array transducers to rotate around the central axis of the spindle;
and wherein, after being excited, the at least two linear array transducers transmit ultrasound waves in a radial direction of the spindle to a first target region in a pelvic cavity and receive returned ultrasound echoes to obtain at least two groups of ultrasound echo data corresponding to the at least two linear array transducers, and the at least two groups of ultrasound echo data are processed to obtain ultrasound images in at least two different longitudinal sections.

2. The intracavitary ultrasound probe of claim 1, wherein the transducer assembly comprises a first linear array transducer and a second linear array transducer, and wherein, an operating frequency range of the first linear array transducer is different from or the same as an operating frequency range of the second linear array transducer, and/or, a size of the first linear array transducer is different from or the same as a size of the second linear array transducer, and/or, a distance from a front end of the first linear array transducer to a front end of the spindle is different from or the same as a distance from a front end of the second linear array transducer to the front end of the spindle.

3. The intracavitary ultrasound probe of claim 1, wherein, the transducer assembly comprises a first linear array transducer and a second linear array transducer, and the first linear array transducer and the second linear array transducer are symmetrically arranged on two side walls of the spindle with respect to a plane passing through the central axis of the spindle.

4. The intracavitary ultrasound probe of claim 1, further comprising a convex array transducer, wherein, the spindle comprises a front end and a rear end extending along an axial direction of the spindle, the driving assembly is connected to the rear end of the spindle or to a position of the spindle near the rear end, and the convex array transducer is located at the front end of the spindle and is configured to transmit ultrasound waves to a second target region in the pelvic cavity and receive returned ultrasound echoes.

5. The intracavitary ultrasound probe of claim 4, wherein, an end of the acoustic window is provided with an opening, and the convex array transducer is arranged in the opening and sealed.

6. The intracavitary ultrasound probe of claim 4, wherein, the convex array transducer is mounted on the spindle and is able to be rotated about the central axis of the spindle; or, the intracavitary ultrasound probe further comprises a rotating shaft that is able to be rotated independently relative to the spindle and the convex array transducer is connected to the rotating shaft.

7. The intracavitary ultrasound probe of claim 4, wherein, the transducer assembly comprises a hinge shaft, the convex array transducer is hinged to the front end through the hinge shaft and is able to be oscillated around the hinge shaft, and an axis of the hinge shaft is perpendicular to the axis of the spindle.

8. The intracavitary ultrasound probe of claim 7, wherein, the convex array transducer is oscillated around the hinge shaft at an angle greater than 0° and less than or equal to 180°, the convex array transducer, after being excited, transmits ultrasound waves to the second target region in a radial direction of the hinge shaft and receives returned ultrasound echoes to obtain a convex array ultrasound echo data corresponding to the convex array transducer, and the convex array ultrasound echo data is processed to obtain ultrasound images of a tissue of the second target region in a transverse section.

9. The intracavitary ultrasound probe of claim 1, wherein, the housing assembly further comprises a connecting seat with a spindle rotation hole, the connecting seat is connected between the handle housing and the connecting housing, one end of the spindle is rotatably mounted in the spindle rotation hole and is connected to the driving assembly, and the other end of the spindle extends into the acoustic window.

10. The intracavitary ultrasound probe of claim 9, wherein, the transducer assembly further comprises a bearing, the bearing is arranged in the spindle rotation hole and the acoustic window, and the spindle passes through the bearing.

11. The intracavitary ultrasound probe of claim 9, further comprising a limiting assembly, wherein the limiting assembly is mounted on at least one of the handle housing, the probe housing, the connecting seat and the spindle to enable the spindle to rotate at any angle within a range of not less than 720 degrees.

12. The intracavitary ultrasound probe of claim 11, wherein, the limiting assembly comprises a fixing component, a limiting component and at least one connecting component, the fixing component is fixed to the housing assembly, the limiting component is fixed to the spindle, and the connecting component is rotatably mounted on the spindle and arranged between the fixing component and the limiting component.

13. The intracavitary ultrasound probe of claim 1, wherein, the driving assembly comprises a driving component and a first reducer, and the driving component is connected to the spindle through the first reducer.

14. The intracavitary ultrasound probe of claim 13, wherein, the driving assembly further comprises an encoder, and the encoder is arranged on a side of the driving component opposite to the first reducer and configured to detect a rotational speed of the spindle.

15. The intracavitary ultrasound probe of claim 14, wherein, a second reducer is connected between the encoder and the driving component, and a transmission ratio of the second reducer corresponds to a transmission ratio of the first reducer.

16. The intracavitary ultrasound probe of claim 1, wherein, a hollow cavity is formed in the spindle, a conductive layer for signal transmission is disposed in the hollow cavity, and the at least two linear array transducers are connected to a sidewall of the spindle and connected to the conductive layer;
and wherein, the linear array transducer comprises a support frame, an acoustic lens, a backing layer and a piezoelectric layer electrically connected to the conductive layer, the acoustic lens is disposed outside the piezoelectric layer, the piezoelectric layer is disposed on the support frame through the backing layer, and the support frame is connected to the sidewall of the spindle.

17. The intracavitary ultrasound probe of claim 1, wherein the preset direction is an axial direction of the spindle.

18. An ultrasound imaging device, comprising a display, an ultrasound host and an intracavitary ultrasound probe, the intracavitary ultrasound probe being connected to the ultrasound host, wherein,
the intracavitary ultrasound probe comprises a housing assembly, a driving assembly and a transducer assembly having a spindle, the housing assembly comprises a handle housing and a probe housing connected to the handle housing, the transducer assembly is arranged in the probe housing, and the driving assembly is arranged in the handle housing and connected to the spindle to drive the spindle to rotate about a central axis of the spindle;
the transducer assembly comprises at least two linear array transducers, each of which comprises multiple array elements linearly arranged along a preset direction of the spindle, the probe housing is provided with an acoustic window and a connecting housing, an end of the connecting housing facing away from the acoustic window is connected to the handle housing, and the at least two linear array transducers are spaced apart in a circumferential direction of the spindle and arranged in the acoustic window such that the spindle is able to drive the at least two linear array transducers to rotate around the central axis of the spindle;
after being excited, the at least two linear array transducers transmit ultrasound waves in a radial direction of the spindle to a first target region in a pelvic cavity and receive returned ultrasound echoes to obtain at least two groups of ultrasound echo data corresponding to the at least two linear array transducers;
the ultrasound host is configured to process the at least two groups of ultrasound echo data to obtain ultrasound images in at least two different longitudinal sections; and
the display is configured to display the ultrasound images.
